(19) United States
(12) Patent Application Publication
(10) Pub. No.: US 2004/0057961 A1
(43) Pub. Date: Mar. 25, 2004

(54) Human t cell response to mhc-binding motif clusters

(76) Inventor: Anne De Goot, Providence, RI (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/239,103

(22) PCT Filed: Mar. 20, 2001
(86) PCT No.: PCT/US01/08906

(30) Foreign Application Priority Data
Mar. 20, 2000 (US) 60190834

(51) Int. Cl. A61K 48/00; A61K 39/02; C07K 14/35

(52) U.S. Cl. 424/190.1; 514/44; 530/350

(57) ABSTRACT

The invention provides Mycobacterium tuberculosis (Mtb) vaccine candidate peptides. The invention also provides a method for identifying Mtb vaccine candidate peptides as well as vaccines comprising these Mtb candidate peptides.

### STATEMENT REGARDING FEDERALLY SPONSORED RESEARCH

This invention was made with United States Government support from the National Institutes of Health (NIH Grant No. R01-AI3571). The Government may have certain rights in the invention.

### TECHNICAL FIELD OF INVENTION

This invention relates generally to vaccines and to computer-based algorithms used to predict epitopes.

### BACKGROUND OF THE INVENTION

The reemergence of tuberculosis as a public health issue, particularly Mycobacterium tuberculosis (Mtb) superinfection of Human Immunodeficiency Virus (HIV)-infected individuals, has prompted the need for improvements in vaccination. Recognition of and response to Mycobacterium tuberculosis protein antigens by CD4+ T cells requires the intracellular processing of these antigens, and the subsequent presentation of the derived peptides by class II major histocompatability complex (MHC) molecules at the surface of antigen presenting cells (APC). To identify these T-cell epitopes, the standard approach has been to synthesize overlapping peptides spanning the entire sequence of a given protein antigen. These peptides are then tested for their capacity to stimulate T cell proliferative responses in vitro, using cells from Mtb immune individuals. Although this overlapping peptide method is thorough, it is both cost- and labor-intensive.

The interaction between Mtb protein sequences and the molecules of the immune system (the human leukocyte antigens, “HLA”), which present peptides derived from the proteins of the challenge protein to the immune system and to engage vaccine-trained T cells to respond, can lead to variations in immune responses. Due to the tight-fit nature of the interaction between Mtb-derived peptides and the HLA, changes in amino acid sequence of a challenge strain may interfere with the ability of a given peptide to bind to the HLA molecule, thereby preventing recognition of the challenge strain by T cell clones raised against a vaccine construct.

Sequence modifications at the amino acid level may affect the recognition of the epitope in three ways: (1) by affecting intracellular processing, (2) by interfering with binding (of the peptide) to major histocompatibility (such as major histocompatiblity complex (MHC) or HLA) molecules and presentation of the peptide-HLA complex at the antigen presenting-cell surface, and (3) by interfering with binding of the epitope to the T cell receptor (TCR) (See Germain & Margulies, Ann. Rev. Immunol. 11:403 (1993); Falk et al., Nature 351:290 (1991)).

Computer-based algorithms have been designed to predict T cell epitopes from the amino acid sequences of proteins, and to diminish the cost and labor associated with the identification of T cell epitopes by the overlapping peptide method. See DeGroot, et al., New Generation Vaccines, 2^{nd} Ed. (1996); Meister, et al., Vaccine 13:581 (1995); Roberts, et al., AIDS Res. Hu. Retrovir. 7:593 (1996); Hammer, et al., J. Exp. Med. 180:2353 (1994); Davenport, et al., Immunogenetics 42:392 (1995); Fleckenstein, et al., Eur. J. Biochem. 240:71 (1995). One such algorithm, EpiMer, predicts putative T cell epitopes by searching an amino acid sequence for regions containing clusters of MHC-binding motifs. These “motifs” are defined as recurring amino acid patterns found in a large percentage of peptides that bind to specific MHC alleles.

### SUMMARY OF THE INVENTION

EpiMer is a computer-based algorithm for predicting T-cell epitopes within protein antigens by searching for clusters of major histocompatability complex molecule (MHC) binding motifs. EpiMer was used to identify putative epitopes for four Mycobacterium tuberculosis (Mtb) antigens, 14 kDa, 16 kDa, 19 kDa, and 32 kDa. A total of 23 putative epitopes were predicted, and 28 corresponding peptides were synthesized. Lymphoproliferation assays were conducted using these peptides and peripheral blood mononuclear cells from 40 Mtb-immune and 19 Mtb-naïve subjects recruited from State Tuberculosis Clinic in Providence, R.I.; the Lemuel Shattuck Hospital, Jamaica Plain, Mass.; and the Research Institute of Tropical Medicine, Manila, the Philippines. Of the 28 peptides tested, all were found to induce a proliferative response in at least one Mtb immune individual. Predicted epitopes that contained a higher number of MHC-binding motifs were more likely to stimulate T cell response in a greater number of Mtb immune individuals than those with a lower number of MHC-binding motifs (RR=5.0; 95% confidence intervals 1.7 to 14). There was an increased likelihood of having a proliferative response to a peptide which contained an MHC-binding motif matched for the subject's allele (RR=1.5, 95% CI 0.9 to 2.5).

Algorithms such as EpiMer, which search for regions of MHC-binding motif clustering, may be useful for the development of subunit vaccines against Mtb.

The invention provides Mtb vaccine candidate peptides, including the peptides shown as SEQ ID NOS:1-28. The invention also provides an Mtb vaccine, which is an Mtb peptide in an immunologically acceptable excipient, such as any of the vaccine carriers known in the medical arts. The invention also provides a method for identifying Mtb vaccine candidates that could be presented in the context of more than one HLA.

The details of one or more embodiments of the invention are set forth in the accompanying description. Although any methods and materials similar or equivalent to those described herein can be used in the practice or testing of the invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### BRIEF DESCRIPTION OF THE DRAWINGS

FIG. 1 shows the MHC-binding motif density histograms for the four Mtb protein antigens studied, based on predictions by EpiMer ML 1994. The number of MHC-binding motifs is plotted against the midpoint of an 11 amino acid reading frame. White bars above the motif density histogram indicate peptides tested in other laboratories; epitopes described by these laboratories are indicated in black. All bars below the motif density histogram represent peptides synthesized to correspond to EpiMer predictions; grey bars indicate peptides which also corresponded to published epitopes; black bars indicate when these peptides were also recognized by six or more subjects in the study cohort. FIG. 1(a) shows the 14 kDa Mtb protein; FIG. 1(b) shows the 16 kDa Mtb protein; FIG. 1(c) shows the 19 kDa Mtb protein; and FIG. 1(d) shows the 32 kDa Mtb protein.

FIG. 2 shows the results of lymphoproliferation assays to PPD, TT, PHA, and to peptides performed in Providence, R.I. and Manila, the Philippines. Solid boxes indicate responses of SI≧2 3.0, grey boxes indicate SI≧2.0, open boxes indicate SI<2, N/D indicates the wells with PHA that were not done for that subject. In those cases where response differed between the 1 μg/ml and 10 μg/ml peptide concentration, the data shown are for the higher response. FIG. 2(a) shows the results of the Mtb-immune group (n=40). FIG. 2(b) shows the results of the Mtb-naïve group (n=19).

FIG. 3 is a scatterplot demonstrating the association between the number of motif matches contained within a peptide and the number of Mtb-immune subjects who respond to that peptide.

FIG. 4 is a table containing a “full list” of Mtb peptides.

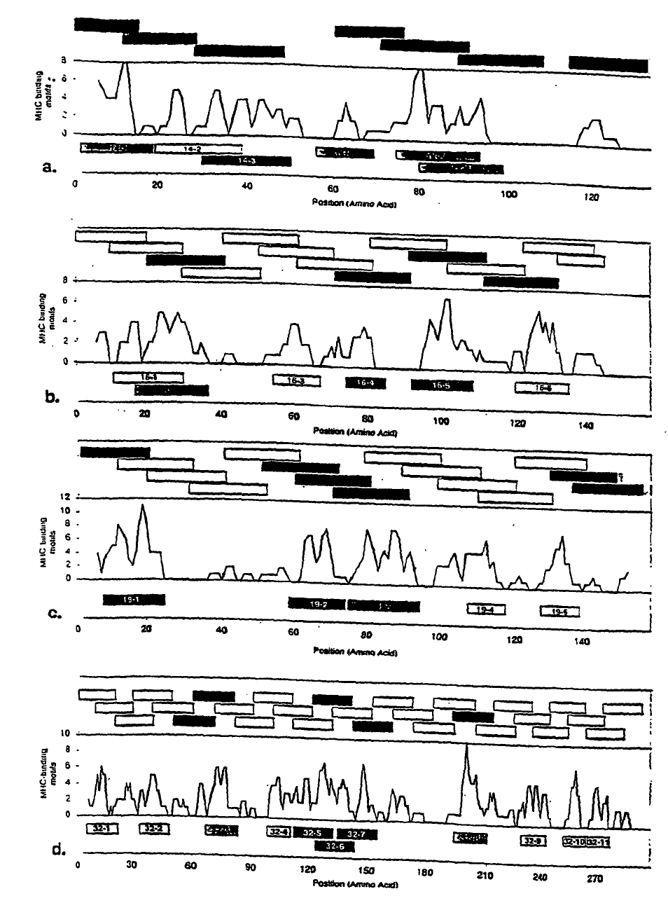

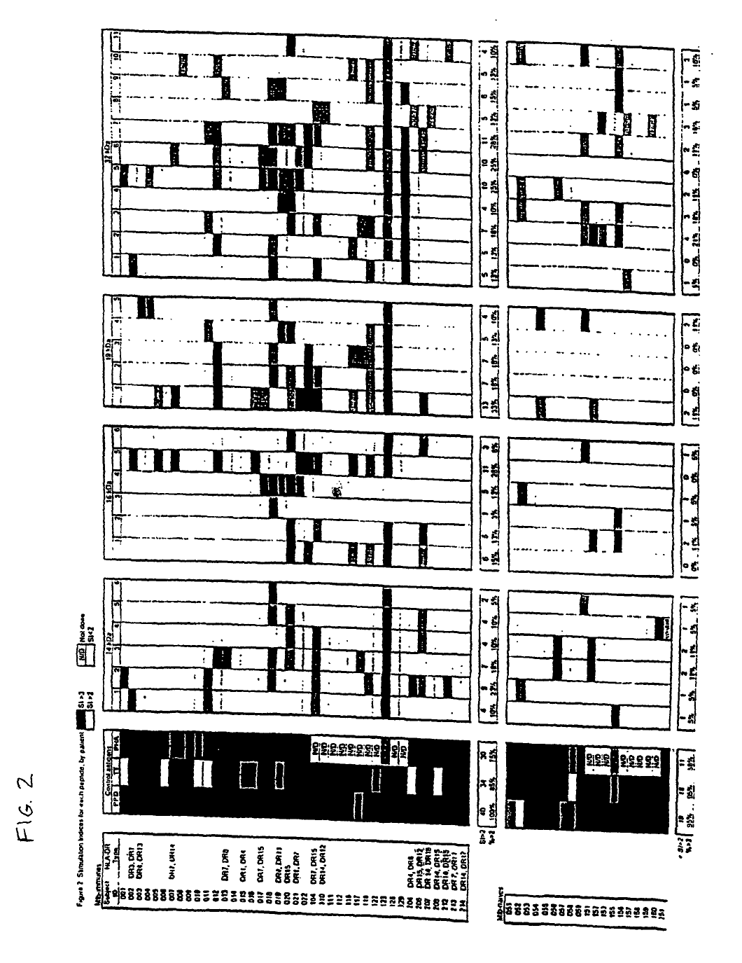

### DETAILED DESCRIPTION OF THE INVENTION

EpiMer, and other MHC-binding motif-based algorithms, may be useful methods for identifying “promiscuous” peptides which can be recognized by a higher number of individuals in outbred human populations. The cost and time savings of this method over the traditional overlapping approach are substantial, and this method may eventually contribute to the development of a novel subunit vaccine against Mtb.

The EpiMer algorithm was applied to four Mtb protein antigens, all of which were selected for analysis because they had been previously shown to stimulate proliferative responses in Mtb-infected subjects. The purpose of this study was to prospectively confirm the utility of the EpiMer algorithm, by (1) measuring the response of Mtb immune subjects to EpiMer-predicted peptides containing clusters of MHC-binding motifs, and by (2) measuring individual responses to other peptides containing motifs matched to the subjects' HLA-DR allele.

Vaccines can include any one of the Mtb vaccine candidate peptides disclosed below, either alone, in combination with suitable carriers, linked to carrier proteins, or expressed from a polynucleotide, such as a “naked DNA” vaccine. The peptides can be administered to a host for treatment of Mtb. The peptides can also be used to enhance immunologic function.

Peptides. The Mtb vaccine candidate peptides can be produced by well known chemical procedures, such as solution or solid-phase peptide synthesis, or semi-synthesis in solution beginning with protein fragments coupled through conventional solution methods, as described by Dugas & Penney, Bioorganic Chemistry, 54-92 (Springer-Verlag, New York, 1981). For example, peptides can be synthesized by solid-phase methodology utilizing a PE-Applied Biosystems 430A peptide synthesizer (commercially available from Applied Biosystems, Foster City, Calif.) and synthesis cycles supplied by Applied Biosystems. Boc amino acids and other reagents are commercially available from PE-Applied Biosystems and other chemical supply companies. Sequential Boc chemistry using double couple protocols are applied to the starting p-methyl benzhydryl amine resins for the production of C-terminal carboxamides. After synthesis and cleavage, purification is accomplished by reverse-phase C18 chromatography (Vydac) column in 0.1% TFA with a gradient of increasing acetonitrile concentration. The solid phase synthesis could also be accomplished using the FMOC strategy and a TFA/scavenger cleavage mixture.

When produced by conventional recombinant means, the Mtb vaccine candidate peptide can be isolated either from the cellular contents by conventional lysis techniques or from cell medium by conventional methods, such as chromatography (see, e.g., Sambrook et al., Molecular Cloning. A Laboratory Manual, 2d Edition (Cold Spring Harbor Laboratory, New York (1989).

In one embodiment, the Mtb vaccine candidate peptide as a maximum size of 50 amino acids in length and a minimum size of 8 amino acids to 11 amino acids (for the relevant SEQ ID NOS). The peptide can be any size between the minimum to maximum size, and one Mtb vaccine candidate peptide can be of a given size independently of another Mtb vaccine candidate peptide. For example, one Mtb vaccine candidate peptide can be 25 amino acids in length while another Mtb vaccine candidate peptide is 45 amino acids in length.

Peptides as antigens. The Mtb vaccine candidate peptides are useful as antigens for raising anti-Mtb immune responses, such as T cell responses (cytotoxic T cells or T helper cells). An “antigen” is a molecule or a portion of a molecule capable of stimulating an immune response, which is additionally capable of inducing an animal or human to produce antibody capable of binding to an epitope of that antigen. An “epitope” is that portion of any molecule capable of being recognized by and bound by an MHC molecule and recognized by a T cell or bound by an antibody. An antigen can have one or more than one epitope. The specific reaction indicates that the antigen will react, in a highly selective maimer, with its corresponding MHC and T cell, or antibody and not with the multitude of other antibodies which can be evoked by other antigens.

A peptide is “immunologically reactive” with an T cell or antibody when it binds to an MHC and is recognized by a T cell or binds to an antibody due to recognition (or the precise fit) of a specific epitope contained within the peptide. Immunological reactivity can be determined by measuring T cell response in vitro or by antibody binding, more particularly by the kinetics of antibody binding, or by competition in binding using as competitors a known peptides containing an epitope against which the antibody or T cell response is directed. The techniques for determining whether a peptide is immunologically reactive with a T cell or with an antibody are known in the art. The peptides can be screened for efficacy by in vitro and in vivo assays. Such assays employ immunization of an animal, e.g., a rabbit or a primate, with the peptide, and evaluation of titers antibody to Mtb or to synthetic detector peptides corresponding to variant Mtb sequences. Methods of determining the spatial conformation of amino acids are known in the art, and include, for example, x-ray crystallography and 2-dimensional nuclear magnetic resonance.

Polynucleotides encoding the peptides. Polynucleotides can encode Mtb vaccine candidate peptides, including peptides fused to carrier proteins. Mtb vaccine candidate peptides can be encoded by either a synthetic or recombinant polynucleotide. The term “recombinant” refers to the molecular biological technology for combining polynucleotides to produce useful biological products, and to the polynucleotides and peptides produced by this technology. The polynucleotide can be a recombinant construct (such as a vector or plasmid) which contains the polynucleotide encoding the Mtb vaccine candidate peptide or fusion protein under the operative control of polynucleotides encoding regulatory elements such as promoters, termination signals, and the like. “Operatively linked” refers to a juxtaposition wherein the components so described are in a relationship permitting them to function in their intended manner. A control sequence operatively linked to a coding sequence is ligated such that expression of the coding sequence is achieved under conditions compatible with the control sequences. “Control sequence” refers to polynucleotide sequences which are necessary to effect the expression of coding and non-coding sequences to which they are ligated. Control sequences generally include promoter, ribosomal binding site, and transcription termination sequence. In addition, “control sequences” refers to sequences which control the processing of the peptide encoded within the coding sequence; these can include, but are not limited to, sequences controlling secretion, protease cleavage, and glycosylation of the peptide. The term “control sequences” is intended to include, at a minimum, components whose presence can influence expression, and can also include additional components whose presence is advantageous, for example, leader sequences and fusion partner sequences. A “coding sequence” is a polynucleotide sequence which is transcribed and translated into a polypeptide. Two coding polynucleotides are “operably linked” if the linkage results in a continuously translatable sequence without alteration or interruption of the triplet reading frame. A polynucleotide is operably linked to a gene expression element if the linkage results in the proper function of that gene expression element to result in expression of the Mtb vaccine candidate coding sequence. “Transformation” is the insertion of an exogenous polynucleotide (i.e., a “transgene”) into a host cell. The exogenous polynucleotide is integrated within the host genome. A polynucleotide is “capable of expressing” a Mtb vaccine candidate peptide if it contains nucleotide sequences which contain transcriptional and translational regulatory information and such sequences are “operably linked” to polynucleotide which encode the Mtb vaccine candidate peptide. A polynucleotide that encodes a peptide coding region can be then amplified, for example, by preparation in a bacterial vector, according to conventional methods, for example, described in the standard work Sambrook et al., Molecular Cloning. A Laboratory Manual (Cold Spring Harbor Press 1989). Expression vehicles include plasmids or other vectors. Prokaryotic vectors known in the art include plasmids such as those capable of replication in E. coli (such as, for example, pBR322, ColE1, pSC101, pACYC184, BVX).

The polynucleotide encoding the Mtb vaccine candidate peptide can be prepared by chemical synthesis methods or by recombinant techniques. The polypeptides can be prepared conventionally by chemical synthesis techniques, such as described by Merrifield, J. Amer. Chem. Soc. 85:2149 (1963). See also, Stemmer et al., Gene 164:49 (1995). Synthetic genes, the in vitro or in vivo transcription and translation of which will result in the production of the protein can be constructed by techniques well known in the art (see Brown et al., Methods in Enzymology 68:109 (1979)). The coding polynucleotide can be generated using conventional DNA synthesizing apparatus such as the Applied Biosystems Model 380A or 380B DNA synthesizers (commercially available from Applied Biosystems, Inc., 850 Lincoln Center Drive, Foster City, Calif. 94404).

Alternatively, systems for cloning and expressing Mtb vaccine candidate peptides include various microorganisms and cells which are well known in recombinant technology. These include, for example, various strains of E. coli, Bacillus, Streptomyces, and Saccharomyces, as well as mammalian, yeast and insect cells. Suitable vectors are known and available from private and public laboratories and depositories and from commercial vendors. See, Sambrook et al., Molecular Cloning, A Laboratory Manual (Cold Spring Harbor Press 1989). See also PCT International patent application WO 94/01139). These vectors permit infection of patient's cells and expression of the synthetic gene sequence in vivo or expression of it as a peptide or fusion protein in vitro.

Polynucleotide gene expression elements useful for the expression of cDNA encoding peptides include, but are not limited to (a) viral transcription promoters and their enhancer elements, such as the SV40 early promoter, Rous sarcoma virus LTR, and Moloney murine leukemia virus LTR; (b) splice regions and polyadenylation sites such as those derived from the SV40 late region; and (c) polyadenylation sites such as in SV40. Recipient cells capable of expressing the Mtb vaccine candidate gene product are then transfected. The transfected recipient cells are cultured under conditions that permit expression of the Mtb vaccine candidate gene products, which are recovered from the culture. Host mammalian cells, such as Chinese Hamster ovary cells (CHO) or COS-1 cells, can be used. These hosts can be used in connection with poxvirus vectors, such as vaccinia or swinepox. Suitable non-pathogenic viruses, which can be engineered to carry the synthetic gene into the cells of the host include poxviruses, such as vaccinia, adenovirus, retroviruses and the like. A number of such non-pathogenic viruses are commonly used for human gene therapy, and as carrier for other vaccine agents, and are known and selectable by one of skill in the art. The selection of other suitable host cells and methods for transformation, culture, amplification, screening and product production and purification can be performed by one of skill in the art by reference to known techniques (see, e.g., Gething & Sambrook, Nature 293:620 (1981)). Another preferred system includes the baculovirus expression system and vectors.

The polynucleotide encoding the Mtb vaccine candidate peptide can be used in a variety of ways. For example, a polynucleotide can express the Mtb vaccine candidate peptide in vitro in a host cell culture. The expressed Mtb vaccine candidate peptide immunogens, after suitable purification, can then be incorporated into a pharmaceutical reagent or vaccine.

Alternatively, the polynucleotide encoding the Mtb vaccine candidate peptide immunogen can be administered directly into a human as so-called “naked DNA” to express the peptide immunogen in vivo in a patient. (see, Cohen, Science 259:1691 (1993); Fynan et al., Proc. Natl. Acad. Sci. USA, 90:11478 (1993); and Wolff et al., BioTechniques 11:474 (1991)). The polynucleotide encoding the Mtb vaccine candidate peptide immunogen can be used for direct injection into the host. This results in expression of the Mtb vaccine candidate peptide by host cells and subsequent presentation to the immune system to induce anti-Mtb antibody formation in vivo.

Determinations of the sequences for the polynucleotide coding region that codes for the Mtb vaccine candidate peptides described herein can be performed using commercially available computer programs, such as DNA Strider and Wisconsin GCG. Owing to the natural degeneracy of the genetic code, the skilled artisan will recognize that a sizable yet definite number of DNA sequences can be constructed which encode the claimed peptides (see, Watson et al., Molecular Biology of the Gene, 436-437 (the Benjamin/Cummings Publishing Co. 1987)).

Treatment of Mtb infection. The method for reducing the levels of Mtb involves exposing a human to a Mtb vaccine candidate peptides, actively inducing antibodies that react with Mtb, and impairing the multiplication of Mtb in vivo. This method is appropriate for an Mtb infected subject with a competent immune system, or an uninfected or recently infected subject. The method induces antibodies, which react with Mtb, which reduces multiplication during any initial acute infection with Mtb.

The terms “treating,” “treatment,” and the like are used herein to mean obtaining a desired pharmacologic or physiologic effect. The effect can be prophylactic in terms of completely or partially preventing a disorder or sign or symptom thereof, or can be therapeutic in terms of a partial or complete cure for a disorder and/or adverse effect attributable to the disorder. “Treating” as used herein covers any treatment and includes: (a) preventing a disorder from occurring in a subject that can be predisposed to a disorder, but has not yet been diagnosed as having it; (b) inhibiting the disorder, i.e., arresting its development; or (c) relieving or ameliorating the disorder. An “effective amount” or “therapeutically effective amount” is the amount sufficient to obtain the desired physiological effect. An effective amount of the Mtb vaccine candidate peptide or vector expressing Mtb vaccine candidate peptides is generally determined by the physician in each case on the basis of factors normally considered by one skilled in the art to determine appropriate dosages, including the age, sex, and weight of the subject to be treated, the condition being treated, and the severity of the medical condition being treated. Among such patients suitable for treatment with this method are Mtb infected patients.

Method of administration. Mtb vaccine candidate peptides can be administered in a variety of ways, orally, topically, parenterally e.g. subcutaneously, intraperitoneally, by viral infection, intravascularly, etc. Depending upon the manner of introduction, the Mtb vaccine candidate peptides can be formulated in a variety of ways. The concentration of Mtb vaccine candidate peptides in the formulation can vary from about 0.1-100 wt. %.

The amount of the Mtb vaccine candidate peptide or polynucleotides of the invention present in each vaccine dose is selected with regard to consideration of the patient's age, weight, sex, general physical condition and the like. The amount of Mtb vaccine candidate peptide required to induce an immune response, preferably a protective response, or produce an exogenous effect in the patient without significant adverse side effects varies depending upon the pharmaceutical composition employed and the optional presence of an adjuvant. Generally, for the compositions containing Mtb vaccine candidate peptide, each dose will comprise between about 50 μg to about 1 mg of the Mtb vaccine candidate peptide immunogens/ml of a sterile solution. A more preferred dosage can be about 200 μg of Mtb vaccine candidate peptide immunogen. Other dosage ranges can also be contemplated by one of skill in the art. Initial doses can be optionally followed by repeated boosts, where desirable. The method can involve chronically administering the Mtb vaccine candidate peptide composition. For therapeutic use or prophylactic use, repeated dosages of the immunizing compositions can be desirable, such as a yearly booster or a booster at other intervals. The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.01 to 100 mg/kg of body weight. Ordinarily 1.0 to 5, and preferably 1 to 10 mg/kg/day given in divided doses 1 to 6 times a day or in sustained release form is effective to obtain desired results.

The Mtb vaccine candidate peptide can be employed in chronic treatments for subjects at risk of acute infection. A dosage frequency for such “acute” infections may range from daily dosages to once or twice a week intravenously or intramuscularly, for a duration of about 6 weeks. The peptides can also be employed in chronic treatments for infected patients. In infected patients, the frequency of chronic administration can range from daily dosages to once or twice a week i.v. or i.m., and may depend upon the half-life of the immunogen (e.g., about 7-21 days). However, the duration of chronic treatment for such infected patients is anticipated to be an indefinite, but prolonged period.

For such therapeutic uses, the Mtb vaccine candidate peptide formulations and modes of administration are substantially identical to those described specifically above and can be administered concurrently or simultaneously with other conventional therapeutics.

Immunologically acceptable carrier. Mtb vaccine candidate peptides can be administered either as individual therapeutic agents or in combination with other therapeutic agents. Mtb vaccine candidate peptides can be administered alone, but are generally administered with a pharmaceutical carrier selected on the basis of the chosen route of administration and standard pharmaceutical practice. The vaccine can further comprise suitable, i.e., physiologically acceptable, carriers—preferably for the preparation of injection solutions—and further additives as usually applied in the art (stabilizers, preservatives, etc.), as well as additional drugs. The patients can be administered a dose of approximately 1 to 10 μg/kg body weight, preferably by intravenous injection once a day. For less threatening cases or long-lasting therapies the dose can be lowered to 0.5 to 5 μg/kg body weight per day. The treatment can be repeated in periodic intervals, e.g., two to three times per day, or in daily or weekly intervals, depending on the status of Mtb infection or the estimated threat of an individual of getting Mtb infection.

For parenteral administration, peptides of the invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques. Suitable pharmaceutical carriers are described in the most recent edition of Remington's Pharmaceutical Sciences, a standard reference text in this field of art. For example, a parenteral composition suitable for administration by injection is prepared by dissolving 1.5% by weight of active ingredient in 0.9% sodium chloride solution. The preparation of these pharmaceutically acceptable compositions, having appropriate pH isotonicity, stability and other conventional characteristics is within the skill of the art.

The vaccine composition can include as the active agents, one of the following above-described components: (a) an Mtb vaccine candidate peptide immunogen, which can be in the form of recombinant proteins or, alternatively, can be in the form of a mixture of carrier protein conjugates; (b) a polynucleotide encoding a Mtb vaccine candidate; (c) a recombinant virus carrying the synthetic gene or molecule; and (d) a bacteria carrying the Mtb vaccine candidate. The selected active component is present in a pharmaceutically acceptable carrier, and the composition can also contain additional ingredients.

Formulations containing the Mtb vaccine candidate peptide can contain other active agents, such as adjuvants and immunostimulatory cytokines, such as IL-12 and other well-known cytokines, for the peptide compositions.

Suitable pharmaceutically acceptable carriers for use in an immunogenic composition are well known to those of skill in the art. Such carriers include, for example, saline, a selected adjuvant, such as aqueous suspensions of aluminum and magnesium hydroxides, liposomes, oil in water emulsions, and others.

Carrier protein. Mtb vaccine candidate peptide immunogens can be linked to a suitable carrier in order to improve the efficacy of antigen presentation to the immune system. Such carriers can be, for instance, organic polymers. A carrier protein can enhance the immunogenicity of the peptide immunogen. Such a carrier can be a larger molecule, which has an adjuvant effect. Exemplary conventional protein carriers include, keyhole limpet hemocyan, E. coli DnaK protein, galactokinase (galK, which catalyzes the first step of galactose metabolism in bacteria), ubiquitin, α-mating factor, β-galactosidase, and influenza NS-1 protein. Toxoids (i.e., the sequence which encodes the naturally occurring toxin, with sufficient modifications to eliminate its toxic activity) such as diphtheria toxoid and tetanus toxoid can also be employed as carriers. Similarly a variety of bacterial heat shock proteins, e.g., mycobacterial hsp-70 can be used. Glutathione reductase (GST) is another useful carrier. One of skill in the art can readily select an appropriate carrier.

Viruses can be modified by recombinant DNA technology such as, e.g. rhinovirus, poliovirus, vaccinia, or influenzavirus, etc. The peptide can be linked to a modified, i.e., attenuated or recombinant virus such as modified influenza virus or modified hepatitis B virus or to parts of a virus, e.g., to a viral glycoprotein such as, e.g., hemagglutinin of influenza virus or surface antigen of hepatitis B virus, in order to increase the immunological response against Mtb-infected cells.

The Mtb vaccine candidate peptides can be in fusion proteins, wherein they are linked to a suitable carrier which might be a recombinant or attenuated virus or a part of a virus such as, e.g., the hemagglutinin of influenza virus or the surface antigen of hepatitis B virus, or another suitable carrier including other viral surface proteins, e.g., surface proteins of rhinovirus, poliovirus, sindbis virus, coxsackievirus, etc., for efficient presentation of the antigenic site(s) to the immune system. In some cases, the antigenic fragments might, however, also be purely, i.e., without attachment to a carrier, applied in an analytical or therapeutical program.

Naked DNA vaccine. Alternatively, polynucleotides can be designed for direct administration as “naked DNA”. Suitable vehicles for direct DNA, plasmid polynucleotide, or recombinant vector administration include, without limitation, saline, or sucrose, protamine, polybrene, polylysine, polycations, proteins, calcium phosphate, or spermidine. See e.g, PCT International patent application WO 94/01139. As with the immunogenic compositions, the amounts of components in the DNA and vector compositions and the mode of administration, e.g., injection or intranasal, can be selected and adjusted by one of skill in the art. Generally, each dose will comprise between about 50 μg to about 1 mg of immunogen-encoding DNA per ml of a sterile solution.

For recombinant viruses containing the coding polynucleotide, the doses can range from about 20 to about 50 ml of saline solution containing concentrations of from about 1×10⁷ to 1×10¹⁰ pfu/ml recombinant virus of the invention. One human dosage is about 20 ml saline solution at the above concentrations. However, it is understood that one of skill in the art can alter such dosages depending upon the identity of the recombinant virus and the make-up of the immunogen that it is delivering to the host.

The amounts of the commensal bacteria carrying the synthetic gene or molecules to be delivered to the patient will generally range between about 10³ to about 10¹² cells/kg. These dosages, will of course, be altered by one of skill in the art depending upon the bacterium being used and the particular composition containing immunogens being delivered by the live bacterium.

Antibodies. An antibody directed against an Mtb vaccine candidate peptide is also an aspect of this invention. Polyclonal antibodies are produced by immunizing a mammal with a peptide immunogen. Suitable mammals include primates, such as monkeys, smaller laboratory animals, such as rabbits and mice; as well as larger animals, such as horse, sheep, and cows. Such antibodies can also be produced in transgenic animals. However, a desirable host for raising polyclonal antibodies to a composition of this invention includes humans. The polyclonal antibodies raised are isolated and purified from the plasma or serum of the immunized mammal by conventional techniques. Conventional harvesting techniques can include plasmapheresis, among others. Such polyclonal antibodies can themselves be employed as pharmaceutical compositions of this invention Alternatively, other forms of antibodies can be developed using conventional techniques, including monoclonal antibodies, chimeric antibodies, humanized antibodies and fully human antibodies See, e.g., U.S. Pat. No. 4,376,110; Ausubel et al., Current Protocols in Molecular Biology (Greene Publishing Assoc. and Wiley Interscience, New York, 1992); Harlow & Lane, Antibodies: a Laboratory Manual, (Cold Spring Harbor Laboratory, 1988); Queen et al., Proc. Nat'l. Acad. Sci. USA 86:10029 (1989); Hodgson et al., Bio/Technology 9:421 (1991); PCT International patent application WO 92/04381 and PCT International patent application WO 93/20210. Other antibodies can be developed by screening hybridomas or combinatorial libraries, or antibody phage displays (Huse et al., Science 246:1275 (1988)) using the polyclonal or monoclonal antibodies produced according to this invention and the amino acid sequences of the primary or optional immunogens.

The term “antibody” includes polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof; provided by any known technique, such as, but not limited to enzymatic cleavage, peptide synthesis or recombinant techniques. An “antigen binding region” is that portion of an antibody molecule which contains the amino acid residues that interact with an antigen and confer on the antibody its specificity and affinity for the antigen. The antibody region includes the framework amino acid residues necessary to maintain the proper conformation of the antigen-binding residues.

Computer Implementation Aspects of the invention may be implemented in hardware or software, or a combination of both. However, preferably, the algorithms and processes of the invention are implemented in one or more computer programs executing on programmable computers each comprising at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system In any case, the language may be a compiled or interpreted language.

Each such computer program is preferably stored on a storage media or device (e.g., ROM, CD-ROM, tape, or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

The following EXAMPLES are presented in order to more fully illustrate the preferred embodiments of the invention These examples should in no way be construed as limiting the scope of the invention, as defined by the appended claims.

### EXAMPLE 1

### Predicting T-Cell Epitopes Within Mtb Protein Antigens

Mtb Antigens. The EpiMer algorithm was applied for Mtb protein antigens, all of which were selected for analysis because they had been previously shown to stimulate proliferative responses in Mtb infected subjects. The purpose of this study was to prospectively confirm the utility of the EpiMer algorithm, by (1) measuring the response of Mtb immune subjects to EpiMer-predicted peptides containing clusters of MHC-binding motifs, and by (2) measuring individual responses to other peptides containing motifs matched to the subjects' LA-DR allele.

Four MW protein antigens were studied: 14 kDa, 16 kDa, 19 kDa, and 32 kDa. The 14 kDa protein, also known as MTP40, is unique to Mtb (See Parra, et al., Infect and Immun 59:3411 (1991); Falla, et al., Infect and Immun 59:2285 (1991)). Falla et al. have identified both B and T cell epitopes within this protein. The 16 kDa protein, the major protein associated with membrane preparations of Mtb, has approximately 30% homology with the alpha-crystallin family of low molecular weight heat shock proteins (See Lee, et al., Infect and Immun 60:2285 (1991); Verbon, et al., J Bacteriol 174:1352 (1992)). Vordermeier, et al. have identified both murine and human T cell epitopes within this protein using the overlapping peptide method (See Vordermeier, et al., Immunology 79:8 (1993); Lamb, et al., Eur J Immunol 18:973 (1988)). The 19 kDa antigen has been shown to contain both human and murine T cell epitopes in a number of studies See Lamb, et al., Eur J Immunol 18:973 (1988); Ashbridge, et al., J Immunol 148:2248 (1992); Faith, et al., Immunology 74:1 (1991); Rees, et al., Immunology 80:407 (1993); Harris, et al., J Immunol 150:407 (1993). The 32 kDa protein, also known as Antigen 85A is one of a number of secreted proteins referred to as the Antigen 85 complex See Wiker, et al., Microbio Rev 56:648 (1992).

Secreted antigens appear to be a primary target of the protective immune response to Mtb because they are believed to be more readily available to macrophages for antigen processing and peptide presentation, leading to a strong T cell response. See Boesen, et al., Infect and Immun 63:1491 (1995). Studies by Huygen et al. and Launois et al. have described both murine and human T cell epitopes within the 32 kDa antigen (See Huygen, et al., Infect and Immun 82:363 (1994); Launois, et al., Infect and Immun 62:3879 (1994)).

Epitope predictions. Amino acid sequences for the four proteins studied were obtained from the Protein Identification Resource (National Library of Medicine) on-line database; these were A43589 (14 kDa Mtb antigen); A43823 (16 kDa Mtb antigen); 802753 (19 kDa Mtb antigen); and A37024 (32 kDa antigen). The EpiMer algorithm uses MHC-binding motif to generate motif matches from the amino acid sequence of a protein. By stepping a reading frame of length r (set to 11 for these experiments) one amino acid at a time through the protein primary structure, the algorithm determines the motif density d for each peptide of length r within the protein Using a minimum density value dmin, set to the sum of the protein's mean MHC-binding motif density d plus one standard deviation, EpiMer extracts only those motif-dense ‘clusters’ with d∃dmin. Finally, the algorithm uses a ‘threading value’ t, of 10, to link selected clusters of contiguous segments into single peptides, depending on their distance apart in the amino acid sequence. (As an example, t=10 would assure that motif-rich clusters from one to ten amino acids apart would be linked into the same predicted peptide, but that clusters of eleven or more amino acids apart would not be linked into a single prediction. The technique of threading was implemented to avoid the generation of multiple peptides overlapping the same short region of a protein. These clusters of MHC-binding motifs constitute the EpiMer algorithm's predictions for putative T cell epitopes for a protein. A full description of the method has been published (Meister, et al., Vaccine 13:581(1995)).

The EpiMer algorithm was executed in Microsoft Excel v4.0 and v5.0 (Microsoft Corporation, Redmond, Wash.) using a Macintosh Quadra 650 and a PowerMacintosh 7100 (Apple Computer, Inc., Cupertino, Calif.). Two versions of EpiMer were used in these experiments: EpiMer ML 1994 and EpiMer ML 0595. The EpiMer ML 1994 motif database contains a total of 15 distinct class II MHC-binding motifs, as described previously (Meister, et al., Vaccine 13:591 (1995)). This version of EpiMer was used in 1994 to predict the peptides for the experiments. Over the course of time, more MHC-binding motifs were published; EpiMer ML 0595 replaced the earlier version of EpiMer in May, 1995 (Roberts, et al., AIDS Res Hu Retrovir 7:593 (1996)). EpiMer ML 0595 employs a more extensive motif list and modifications of the original motifs, described in Roberts, et al.

Peptide synthesis. A total of 23 putative epitopes were predicted by the EpiMer ML 1994 algorithm from the four Mtb protein antigens studied. In those cases where EpiMer-predicted sequences were greater than 20 amino acids in length, overlapping peptides were identified that spanned the given EpiMer prediction. This was necessary due to difficulties encountered in the synthesis of peptides greater than 20 amino acids in length Four cases arose in which the synthesis of overlapping peptides was necessary. In three of these cases a 20 amino acid peptide was generated from the first 20 amino acids of the predicted epitope's sequence, and another with the sequence of the last 20 amino acids of the same prediction (see peptides 14-2, 14-3 [overlap of 9 amino acids]; 14-5, 14-6 [overlap of 14 amino acids]; and 16-1, 16-2 [overlap of 14 amino acids]). In one case, an EpiMer-prediction included 42 amino acids; three overlapping 20 amino acid peptides were synthesized to span the length of this predicted putative epitope (peptides 32-5, 32-6, 32-7). A total of 28 peptides were synthesized to correspond to the 23 putative epitopes predicted by EpiMer at the Torrey Pines Institute (San Diego, Calif.), using the simultaneous multiple peptide synthesis method, employing t-butoxycarbonyl amino acids and hydrogen fluoride cleavage. The purity of these synthetic peptides was determined by high-pressure liquid chromatography and mass spectroscopy.

### EXAMPLE 2

### Measuring Response of Mtb Immune Subjects to EpiMer-Predicted Peptides

Subjects. Forty purified protein derivative (PPD) skin-test positive subjects (designated Mtb immune for this study) were recruited from the Roger Williams Hospital Tuberculosis Clinic, Providence, R.I.; the Lemuel Shattuck Hospital, Jamaica Plain, Mass.; and the Research Institute for Tropical Medicine, Manila, the Philippines. All of these subjects had skin induration greater than 10 mm at 48 hours post inoculation with 5 U.S. Units (TU) PPD (Connaught Laboratories, Ontario, Canada), and all were healthy adults (ages ranged from 18 to 66 years) with no radiologic or clinical sign of active TB.

Nineteen PPD skin-test negative subjects were recruited to serve as controls (Mtb näive group). For this group of subjects, the ages ranged from 21 to 46 years. None of these subjects had been immunized with the BCG vaccine. None of the subjects in either group gave a history of TB exposure, HIV infection, or of taking immunosuppressive medications. IRB approval was obtained from the Roger Williams Hospital (TB Clinic); Lemuel Shattuck Hospital; Brown University; and the Research Institute for Tropical Medicine, Manila, the Philippines, and all study subjects gave informed consent to participate in this study.

Cell isolation. Whole blood was collected in heparinized Venoject tubes (Curtin-Matheson Scientific. Wilmington, Mass.). Peripheral blood mononuclear cells (PBMG) were isolated by centrifugation over Histopaque 1.077 (Sigma Chemical Corp., St. Louis, Mo.). Cells were suspended in RPMI 1640 medium (JRH Biosciences, Lenexa, Kans.) supplemented with 2 mM L-glutamine (Sigma), 5 μg/ml of cefazolin (Sigma), and 10% heat-inactivated human AB serum (Sigma).

Lymphoproliferation assay. Assays were performed in 96-well round bottom plates (Corning-Costar Corp., Cambridge, Mass.). Triplicate cultures containing 2×10⁵ PBMC in 0.2 ml culture medium, with or without peptide, were incubated for 4 days at 37° C. in a 5% CO₂-enriched, humidified atmosphere. After this 4 day period, 1 μCi of [³H]thymidine (ICN Biomedicals, Costa Mesa, Calif.) was added to each well for an additional 14 hours of incubation. Responses to several control antigens including PPD (5 μg/ml) (Connaught Laboratories, Inc., Swiftwater, Pa.); tetanus toxoid (TT) (5 μg/ml) (Connaught); phytohemaglutinin (PHA), a nonspecific T cell mitogen, (1 μg/ml) (Sigma); and to each of the 28 EpiMer-predicted peptides (1=g/ml and 10=g/ml) were measured. After incubation with [³H]thymidine, the 96-well plates were harvested onto fiberglass filtermats using a 96-well semiautomated Tomtec Mach IIM harvester (Tomtec, Groton, Conn.). Counts per minute (cpm) were measured in a Betaplate 1205 scintillation counter (Wallac, Turku, Finland). Stimulation indices (SI) were calculated as follows:

$$SI = \frac{mean ( cpm in wells containing peptide or antigen )}{mean ( cpm in wells containing medium and cells alone )}$$

Responses to peptides were graded as positive if SI≧2.0.

Statistical analysis. All statistical analyses were performed in Microsoft Excel v4.0 or v5.0 (Microsoft Corporation, Redmond, Wash.) using a Macintosh Quadra 650 or a PowerMacintosh 7100 (Apple Computer, Inc., Cupertino, Calif.). The relative risk (RR) of response and the 95% confidence intervals (CI) for this ratio were calculated using an Excel v4.0 spreadsheet.

Results.

Epitope predictions. EpiMer-predicted epitopes are shown in Tables 1 and 2. Table 3 lists 26 distinct MHC-binding motifs described for the human leukocyte antigen (BA) class II alleles and included in the EpiMer ML 0595 motif list at the time these experiments were initiated. In some cases, multiple, distinct MHC-binding motifs have been published for the same HLA allele. In both versions of EpiMer used here, each match to a motif was counted separately and equally.

TABLE 1

| EpiMer predictions: sequences, previously published epitopes and putative MHC binding motifs | | | | | | | | | | |
|---|---|---|---|---|---|---|---|---|---|---|
| | | | | | | | Prov | Prov | Phil | Phil |
| | | | | | Alleles | # of Motifs | # | % | # | % |
| | | | | # | represented | (ML 0595)^{V} | responders | responders | responders | responders |
| Identifier | Start | Stop | Sequence | aa's | (putative) | (putative) | (n = 22) | (n = 22) | (n = 38) | (n = 38) |
| 14-1* | 1 | 18 | MLGNAPSVVPNTTLGMHC | 18 | DQ3.1, DR1(c), | 8 | 2 | 5% | 2 | 5% |
| | | | (SEQ ID NO: 1) | | DRB1*0101(R) | | | | | |
| | | | | | DRB1*0301, | | | | | |
| | | | | | DRB1*0701 | | | | | |
| 14-2 | 19 | 38 | GSFGSAPSNGWLKLGLVEFG | 20 | DQ3.1, DR1(c), | 5 | 3 | 8% | 6 | 15% |
| | | | (SEQ ID NO: 2) | | DRB1*0701 | | | | | |
| 14-3* | 30 | 49 | LKLGLVEFGGVAKLNAEVMS | 20 | DPw4(b), DQ3.1, | 9 | 4 | 10% | 3 | 8% |
| | | | ( SEQ ID NO: 3) | | HLA-DR1(c), | | | | | |
| | | | | | DRB1*0101(R), | | | | | |
| | | | | | DRB1*1101, | | | | | |
| | | | | | DRB1*1201, | | | | | |
| | | | | | DRB1*1501 | | | | | |
| 14-4 | 56 | 68 | QAVMLGTGTPNRA | 13 | DR1(c) | 1 | 1 | 3% | 3 | 8% |
| | | | (SEQ ID NO: 4) | | | | | | | |
| 14-5* | 74 | 93 | CEVWSNVSETISGPRLYGEM | 20 | DQ3.1, DR1(c), | 6 | 2 | 5% | 2 | 5% |
| | | | (SEQ ID NO: 5) | | DRB1*0101(R), | | | | | |
| | | | | | DPB1*0301, | | | | | |
| | | | | | DRB1*0701, | | | | | |
| | | | | | DRB1*0801 | | | | | |
| 14-6* | 80 | 99 | VSETISGPRLYGEMTMQGTR | 20 | DQ7, | 4 | 1 | 3% | 1 | 3% |
| | | | (SEQ ID NO: 6) | | DRB1*0401(DR4 | | | | | |
| | | | | | Dw4)(a), | | | | | |
| | | | | | DRB1*0404(DR4 | | | | | |
| | | | | | Dw14), | | | | | |
| | | | | | DRB1*0801 | | | | | |
| 16-1 | 11 | 30 | RSLFPEFSELFAAFPSFAGL | 20 | DRB1*0701, | 6 | 2 | 5% | 4 | 10% |
| | | | (SEQ ID NO: 7) | | DRB10101(R), | | | | | |
| | | | | | DR1(c), DPw4(b) | | | | | |
| 16-2* | 17 | 36 | FSELFAAFPSFAGLRPTFDT | 20 | DPw4(b), DQ3.1, | 6 | 1 | 3% | 4 | 10% |

TABLE 2

| | | | | | | #Motifs |
|---|---|---|---|---|---|---|
| | | | | | | ML |
| Peptide | Start | Stop | Sequence | # aa's | Alleles Represented (Putative) | 0595)^{V} |
| 16-4* | 74 | 84 | LTIKAERTEQK (SEQ ID | 11 | — | 0 |
| | | | NO: 10) | | | |
| 32-3* | 67 | 82 | SVVMPVGGQSSFYSDW (SEQ | 16 | — | 0 |
| | | | ID NO: 20) | | | |
| 32-9 | 227 | 242 | KFLEGFVRTSNIKFQD (SEQ | 16 | — | 0 |
| | | | ID No: 26) | | | |
| 14-4 | 56 | 68 | QAVMLGTGTPNPA (SEQ ID | 13 | DR1(c) | 1 |
| | | | NO: 4) | | | |
| 16-3 | 54 | 66 | AELPGVDPDKDVD (SEQ ID) | 13 | DRB5*0101(a) | 1 |
| | | | NO: 9) | | | |
| 19-4 | 107 | 117 | VTLGYTSGTGQ (SEQ ID | 11 | DRB1*0404(DR4Dw14) | 1 |
| | | | NO: 16) | | | |
| 32-10 | 252 | 264 | GVFDFPDSGTHSW (SEQ ID | 13 | DR1(c) | 1 |
| | | | NO: 27) | | | |
| 32-2 | 35 | 47 | ALYLLDGLRAQDD (SEQ ID | 13 | HLA-DRB1*0101(R) | 1 |
| | | | NO: 19) | | | |
| 16-6* | 119 | 135 | GILTVSVAVSEGKPTEK (SEQ | 17 | DQ3.1, DQ7, | 3 |
| | | | ID NO: 12) | | DRB1*0401(DR4Dw4)(a) | |
| 9-5 | 127 | 139 | SHYKITGTATGVD (SEQ ID | 13 | DP9, DPw4(b), DQ3.1 | 3 |
| | | | NO: 17) | | | |
| 32-11 | 265 | 276 | EYWGAQLNAMKP (SEQ ID | 12 | DPA1*0102/DPB1*0201, | 3 |
| | | | NO: 28) | | DQ3.1, DR1(c) | |
| 32-4 | 99 | 110 | TFLTSELPGWLQ (SEQ ID | 12 | HLA-DR1(c), DRB1*0301 | 3 |
| | | | NO: 21) | | | |
| 14-6* | 80 | 99 | VSETISGPRLYGEMTMQGTR | 20 | DQ7, | 4 |
| | | | (SEQ ID NO: 6) | | DRB1*0401(DR4Dw4)(a), | |
| | | | | | DRB1*0404(DR4Dw14), | |
| | | | | | DRB1*0801 | |
| 32-1 | 6 | 20 | LPVEYLQVPSPSMGR (SEQ | 15 | DPA1*0102/DPB1*0201, | 4 |
| | | | ID NO: 18) | | DR1(c), DRB1*0101(R), | |
| | | | | | DRB1*0701 | |
| 14-2 | 19 | 38 | GSFGSAPSNGWLKLGLVEFG | 20 | DQ3.1, DR1(c), DRB1*0701 | 5 |
| | | | (SEQ ID NO: 2) | | | |
| 14-5* | 74 | 93 | CEVWSNVSETISGPRLYGEM | 20 | DQ3.1, DR1(c), | 6 |
| | | | (SEQ ID NO: 5) | | DRB1*0101(R), DRB1*0301, | |
| | | | | | DRB1*0701, DRB1*0801 | |
| 16-1 | 11 | 30 | RSLFPEFSELFAAFPSFAGL | 20 | DRB1*0701, DRB1*0101(R), | 6 |
| | | | (SEQ ID NO: 7) | | DR1(c), DPw4(b) | |
| 16-2* | 17 | 36 | FSELFAAFPSFAGLRPTFDT | 20 | DPw4(b), DQ3.1, DR1(c), | 6 |
| | | | (SEQ ID NO: 5) | | DRB1*0101(R) | |
| 19-2* | 58 | 74 | QNVTGSVVCTTAAGNVN | 17 | DPw4(b), HLA-DQ3.1, HLA- | 6 |
| | | | (SEQ ID NO: 14) | | DR1(c) | |
| 32-7* | 135 | 154 | LAIYHPQQFVYAGAMSGLLD | 20 | DPw4(b), DQ3.1, DR1(c), | 7 |
| | | | (SEQ ID NO: 24) | | DRB1*0101(R), DRB1*0701 | |
| 14-1 | 1 | 18 | MLGNAPSVVPNTILGMHC | 18 | DQ3.1, DR1(c), | 8 |
| | | | (SEQ ID NO: 1) | | DRB1*0101(R), DRB1*0301, | |
| | | | | | DRB1*0701 | |
| 16-5* | 92 | 107 | FAYGSFVRTVSLPVGA (SEQ | 16 | DPw4(b), DQ3.1, DQ7, DR1(c), | 8 |
| | | | ID NO: 11) | | DRB1*0401(DR4Dw4)(a), | |
| | | | | | DRB1*0701 | |
| 32-8* | 193 | 210 | PLLNVGKLIANNTRVWVY | 18 | DQ3.1, DR1(c), | 8 |
| | | | (SEQ ID NO: 25) | | DRB1*0404(DR4Dw14), | |
| | | | | | DRB1*0701, DRB1*0801, | |
| | | | | | DRB1*1501, DPw4(b) | |
| 14-3* | 30 | 49 | LKLGLVEFGGVAKLNAEVMS | 20 | DPw4(b), DQ3.1, HLA-DR1(c), | 9 |
| | | | (SEQ ID NO: 3) | | DRB1*0101(R), DRB1*1101, | |
| | | | | | DRB1*1201, DRB1*1501 | |
| 32-5* | 113 | 132 | RHVKPTGSAVVGLSMAASSA | 20 | DQ3.1, DR1(c), | 9 |
| | | | (SEQ ID N0: 22) | | DRB1*0401(DR4Dw4)(a), | |
| | | | | | DRB1*0404(DR4dDw14), | |
| | | | | | DRB1*0701, DPw4(b) | |
| 19-3* | 75 | 94 | LAIGGAATGIAAVLTDGNPP | 20 | DPw4(b), DQ3.1, DR1(c), | 12 |
| | | | (SEQ ID NO: 15) | | DRB1*0101(R) | |
| 19-1* | 7 | 25 | VAVAGAAILVAGLSGCSSN | 19 | DPw4(b), DQ3.1, DR1(c), | 16 |
| | | | (SEQ ID NO: 13) | | DRB1*0101(R), | |
| | | | | | DRB1*0401(DR4Dw4)(a) | |
| 32-6 | 23 | 142 | VGLSMAASSALTLAIYHPQQ | 20 | DPw4(b), DQ3.1, DR1(c), | 16 |
| | | | (SEQ ID NO: 23) | | DRB1*0101(R), | |
| | | | | | DRB1*0401(DR4Dw4)(a) | |
| | | | | | DRB1*0404(DR4Dw14) | |

^{V}= number of motifs is determined by counting the absolute number of MHC binding motifs, including reiterated motifs
*= 15 EpiMer peptides that correlate with published epitopes

TABLE 3
Class II MHC Binding Motifs utilized in Epimer Motif
List 0595

| Motif | | Position in peptide | | | | | | | | | | |
|---|---|---|---|---|---|---|---|---|---|---|---|---|
| Name | Reference(s) | 1+0 | 1+1 | 1+2 | 1+3 | 1+4 | 1+5 | 1+6 | 1+7 | 1+8 | 1+9 | 1+10 |
| HLA- | Dong et al., J | RK | | | | | AGI | | | LV | | |
| DP9 | Immunol | | | | | | | | | | | |
| | 154: 4536-4545 | | | | | | | | | | | |
| | (1995) | | | | | | | | | | | |
| HLA- | Rammensee et al., | FLM | | | | FL | | | IAMV | | | |
| DPA1* | ImmunogeneticS | VW | | | | MY | | | | | | |
| 0102/ | 41: 178-228 (1995) | Y | | | | | | | | | | |
| DPB1* | | | | | | | | | | | | |
| 0201 | | | | | | | | | | | | |
| HLA- | Falk et al., | FLY | | | | | | FLY | | | VYIA | |
| DPw4 | Immunogenetics | MIV | | | | | | MVIA | | | L | |
| (b) | 39: 230-242 | A | | | | | | | | | | |
| | (1994) | | | | | | | | | | | |
| HLA- | Verreck et al., EJI | K | | | I | | | | | F | | |
| DQ2 | 24: 375-379 (1994) | | | | | | | | | | | |
| HLA- | Sidney et el., J. | No | No | AG | No | AV | | | | | | |
| DQ3.1 | Immunol. | RK | RK | ST | DE | LI | | | | | | |
| | 152: 4518-4525 | DEP | DE | | | | | | | | | |
| | (1994) | | | | | | | | | | | |
| HLA- | Falk et al., | FYI | | | | VLI | | YFM | | | | |
| DQ7 | Immunogenetics | ML | | | | MY | | LVI | | | | |
| | 39: 230-42 (1994) | V | | | | | | | | | | |
| HLA- | Hammer et al., J. | YF | No | No | ML | No | GA | No | No | LMAI | | |
| DR1 | Exp. Med. 176: | W | DE | DE | | DE | | DE | DE | GTVQ | | |
| (a) | 1007-1013 (1992) | | | | | | | | | S | | |
| HLA- | Kropshofer et al., J. | AVI | | | | | STA | | | AVILY | | |
| DR1 | Exp. Med. | LYF | | | | | VIL | | | FWMC | | |
| (c) | 175: 1799-1803 | WM | | | | | PQ | | | | | |
| | (1992) | C | | | | | | | | | | |
| HLA- | Hammer et al., | YW | R | | ML | GA | | | L | | | |
| DR1 | PNAS USA 91 | FIL | | | | | | | | | | |
| (d) | (10): 4456-4460 | VM | | | | | | | | | | |
| | (1994) | | | | | | | | | | | |
| HLA- | Sidney et al., J. | AVI | | AV | QN | | RK | | | | | |
| DR3 | Immunol. | LYF | | IL | RK | | H | | | | | |
| (b) | 149: 2634-2640 | WM | | YF | DES | | | | | | | |
| | (1992) | C | | W | T | | | | | | | |
| | | | | MC | | | | | | | | |
| HLA- | Rammensee et al., | YVL | | | LAI | | AGS | | | LAIVN | | |
| DRB1 | Immunogenetics | FIA | | | VM | | TP | | | FYM | | |
| *0101 | 41: 178-228 (1995) | MW | | | NQ | | | | | | | |
| (R) | | | | | | | | | | | | |
| HLA- | Corrected. Chicz et | YF | | | DEN | | | | | YMLI | | |
| DRB1 | al., J. Exp. Med. | WLI | | | | | | | | | | |
| *0301 | 178: 27-47 (1993) | VM | | | | | | | | | | |
| HLA- | Malcherek et al., | LIF | | | D | | KRE | | LYF | | | |
| DRB1 | Intl. Immunol. | MV | | | | | QN | | | | | |
| *0301 | 5: 1229-1237 | | | | | | | | | | | |
| (a) | (1994); stretch | | | | | | | | | | | |
| | variant of | | | | | | | | | | | |
| | Rammensee et al. | | | | | | | | | | | |
| | (1995) | | | | | | | | | | | |
| HLA- | Malcherek et al., | LIF | | | D | | KRE | | | LYF | | |
| DPB1 | Intl. Immunol. | MV | | | | | QN | | | | | |
| *0301 | 5: 1229-1237 | | | | | | | | | | | |
| (b) | (1994); Also | | | | | | | | | | | |
| | Rammensee et al. | | | | | | | | | | | |
| | (1995) | | | | | | | | | | | |
| HLA- | Malcherek et al., | LIF | | | D | | KRE | | | | LYF | |
| DRB1 | Intl. Immunol. | MV | | | | | QN | | | | | |
| *0301 | 5: 1229-1237 | | | | | | | | | | | |
| (c) | (1994); stretch | | | | | | | | | | | |
| | variant of | | | | | | | | | | | |
| | Rammensee et al. | | | | | | | | | | | |
| | (1995) | | | | | | | | | | | |
| HLA- | Rammensee et al., | FY | | | FWI | | NST | DAS | | ASQG | | |
| DRB1 | immunogenetics | WIL | | | LVA | | QH | VHPL | | LTVK | | |
| *0401 | 41: 178-228 (1995) | VM | | | DE | | RVL | NMI | | | | |
| (Dr4D | | GA | | | M | | IM | | | | | |
| w4(a)) | | | | | | | | | | | | |
| HLA- | Rammensee et al., | VIL | | | No | | NQS | RKH | | AHGQ | | |
| DRB1 | Immunogenetics | M | | | DE | | TK | NQP | | SNLT | | |
| *0402 | 41: 178-228 (1995) | | | | | | | | | V | | |
| (DR4D | | | | | | | | | | | | |
| w10) | | | | | | | | | | | | |
| HLA- | Rammensee et al., | VIL | | | No | | NTS | ANV | | ASQG | | |
| DRB1 | Immunogenetics | M | | | RK | | QR | QKP | | LTVK | | |
| *0404 | 41: 178-228 (1995) | | | | | | | DMS | | | | |
| (DR4D | | | | | | | | HLIT | | | | |
| w14) | | | | | | | | | | | | |
| HLA- | Rammensee et al., | FY | | | VIL | | NTS | ANV | | DEQ | | |
| DRB1 | lmmunogenetics | WVI | | | MD | | QK | QKP | | | | |
| *0405 | 41: 178-228 (1995) | LM | | | E | | DV | DMS | | | | |
| (DR4D | | | | | | | | HLIT | | | | |
| w15) | | | | | | | | | | | | |
| (a) | | | | | | | | | | | | |
| HLA- | Corrected. Chicz et | WY | | | | | TS | | | WYFM | | |
| DRB1 | al., J. Exp. Med. | FM | | | | | | | | LVI | | |
| *0701 | 178: 27-47 (1993) | VLI | | | | | | | | | | |
| HLA- | Corrected. Chicz et | YF | | | | KR | | | | | | |
| DRB1 | al., J. Exp. Med. | MV | | | | | | | | | | |
| *0801 | 178: 27-47 (1993) | LI | | | | | | | | | | |
| HLA- | Rammensee et al., | WY | | | MLI | | RK | | | | | |
| DRB1 | Immunogenetics | F | | | V | | | | | | | |
| *1101 | 41: 178-228 (1995) | | | | | | | | | | | |
| HLA- | Rammensee et al., | ILF | | LM | | | VYF | | | YFMIV | | |
| DHB1 | Immunogenetics | YV | | NV | | | INA | | | | | |
| *1201 | 41: 178-228 (1995) | | | A | | | | | | | | |
| HLA- | Corrected Chicz et | LIV | | | YF | | | FLIV | | | | |
| DRB1 | al., J. Exp. Med. | | | | WIV | | | M | | | | |
| *1501 | 178: 27-47 (1993) | | | | | | | | | | | |
| HLA- | Rammensee et al., | FYL | | | QVI | | | | | RK | | |
| DRB5 | Immunogenetics | M | | | M | | | | | | | |
| *0101 | 41: 178-228 (1995) | | | | | | | | | | | |
| HLA- | Corrected. Chicz | VFL | | | VMI | | | | KR | | | |
| DRB5 | et al., J. Exp. Med. | M | | | Q | | | | | | | |
| *0101 | 178: 27-47 (1993); | | | | | | | | | | | |
| (a) | adjusted based on | | | | | | | | | | | |
| | Reammensee et al. | | | | | | | | | | | |
| | (1995) | | | | | | | | | | | |

Table 1 lists the 28 peptides (14-1 through 32-11) that were synthesized to correspond to the 23 predicted epitopes, as described in Example 1, supra, and the number of MHC-binding motifs contained within each peptide. Four regions were predicted for the 14 kDa protein, corresponding to six synthesized peptides (14-1 to 14-6); five regions were predicted for the 16 kDa protein, corresponding to six synthesized peptides (16-l to 16-6); five regions were predicted for the 19 kDa protein, corresponding to five synthesized peptides (19-1 to 19-5); and nine regions were predicted for the 32 kDa protein, corresponding to 11 synthesized peptides (32-1 to 32-11).

In Tables 1 and 2, start and stop numbers indicate amino acid positions within the native proteins. Amino acids are abbreviated with their single-letter designations. The alleles represented by the motifs identified in the peptide (from ML 0595) are listed under the heading “Allele”. Under the heading “# of Motif”, the number of binding motifs contained within the given peptide is given, based on the motifs shown in Table 3 (ML 0595). Table 3 is sorted by the number of motif matches, 0-16.

Lymphoproliferation as Table 4 is a list of the Mtb-immune group and the Mtb-näive group. The results of the lymphoproliferation assays for the 28 peptides are listed in FIG. 2. Overall, 29 of 40 (72%) Mtb-immune subjects responded to one or more of the 28 peptides tested. Eleven (28%) of the Mtb-immune subjects failed to respond to any of the 28 peptides.

TABLE 4a
Mtb-immune group

| | | | | Country of | | |
|---|---|---|---|---|---|---|
| Subject | Age | Sex | PPD Status | Origin | BCG | HLA |
| 1 | 27 | F | Unkn date PPD+ | Dominican | Yes | — |
| | | | | Republic | | |
| 2 | 61 | F | Recent convert | USA | No | — |
| 3 | 34 | F | Unkn date PPD+ | Azores | Yes | — |
| 4 | 66 | F | Unkn date PPD+ | USA | No | ✓ |
| 5 | 36 | F | Unkn date PPD+ | Nigeria | Yes | — |
| 6 | 32 | M | Unkn date PPD+ | Columbia | Yes | — |
| 7 | 26 | M | Recent convert | India | Yes | — |
| 8 | 28 | M | Unkn date PPD+ | Mozambique | Yes | — |
| 9 | 34 | F | Recent convert | Haiti | Yes | — |
| 10 | 33 | F | Unkn date PPD+ | USA | No | ✓ |
| 11 | 19 | F | Unkn date PPD+ | Domincan | Yes | ✓ |
| | | | | Republic | | |
| 12 | 35 | M | Unkn date PPD+ | USA | No | — |
| 13 | 38 | F | PPD+ since 1992 | Dominican | Yes | ✓ |
| | | | | Republic | | |
| 14 | 35 | F | Unkn date PPD+ | USA | No | ✓ |
| 15 | 28 | F | PPD+ since 1992 | Cape Verde | Unk | ✓ |
| 16 | 28 | F | Unkn date PPD+ | USA | No | — |
| 17 | 21 | M | Unkn date PPD+ | Dominican | Unk | — |
| | | | | Republic | | |
| 18 | 18 | M | Unkn date PPD+ | Argentina | Yes | — |
| 19 | 33 | M | Unkn date PPD+ | Cape Verde | Yes | ✓ |
| 20 | 53 | F | PPD+ since 1970 | Canada | No | ✓ |
| 21 | 54 | F | PPD+ since 1991 | Israel | Yes | ✓ |
| 22 | 66 | F | PPD+ since 1993 | Phillipines | No | — |

TABLE 4b
Mtb-naive group

| | | | | Country of | | |
|---|---|---|---|---|---|---|
| Subject | Age | Sex | PPD Status | Origin | BCG | HLA |
| 1 | 38 | F | PPD negative | USA | No | [symbol] |
| 2 | 40 | M | PPD negative | USA | No | [symbol] |
| 3 | 22 | F | PPD negative | USA | No | [symbol] |
| 4 | 21 | M | PPD negative | USA | No | [symbol] |
| 5 | 46 | F | PPD negative | USA | No | [symbol] |
| 6 | 28 | M | PPD negative | USA | No | [symbol] |
| 7 | 25 | F | PPD negative | USA | No | [symbol] |

All but six Mtb-immune subjects and one Mtb-naive control showed a proliferative response to TT (SI range 2-182). All Mtb-immune subjects showed an in vitro response to PPD, although the intensity of these in vitro responses varied greatly (SI range 2.2-166; median 20). Of the 41 subjects who were tested, all demonstrated a robust response to phytohemagglutinin.

Of the 28 peptides synthesized, all were found to induce a proliferative response (SI≧2.0) in at least one Mtb-immune individual. The number of Mtb-immune responders varied from one (for peptide 16-3) to 13 (for peptide 19-1). Twelve peptides (14-2, 14-3, 16-1, 16-5, 19-1, 19-2, 19-3, 32-5, 32-6, 32-7, and 32-9) induced a proliferative response in six or more Mtb-immune subjects.

Individual study subjects responses to these twelve broadly-recognized peptides are shown in FIG. 2. In 10 of the 19 (53%) Mtb-naïve individuals, at least one of the 25 peptides induced a proliferative response (SI≧2.0).

Certain peptides were identified to which none of the Mtb-naive controls responded, and to which a high proportion of the Mtb-immune subjects showed a response (16-5, 28% responders and 32-8, 25% responders). Six of the 19 Mtb-naïve controls and 14 of the 40 Mtb-immune subjects showed at least one response to the Mtb-unique 14 kDa peptide. The number of subjects was too small to determine whether there was any relationship between number of responses to peptides and either BCG vaccination status or race/ethnicity; likewise, no relationship could be observed between BCG status or race/ethnicity and response/non-response to a particular peptide.

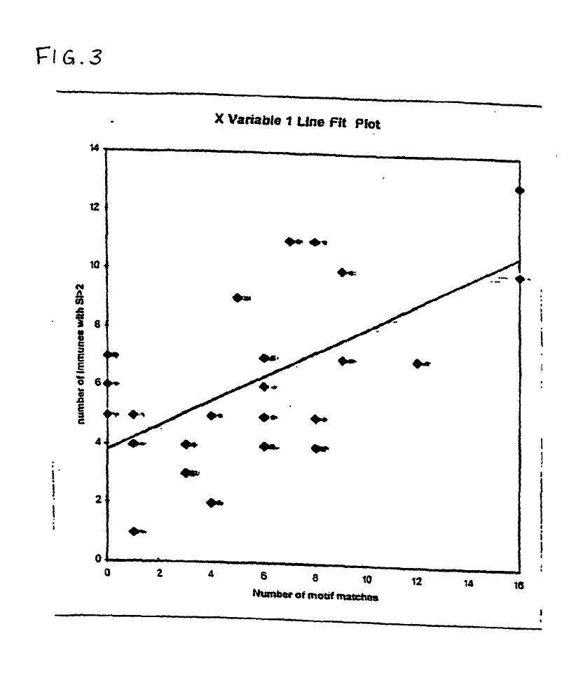

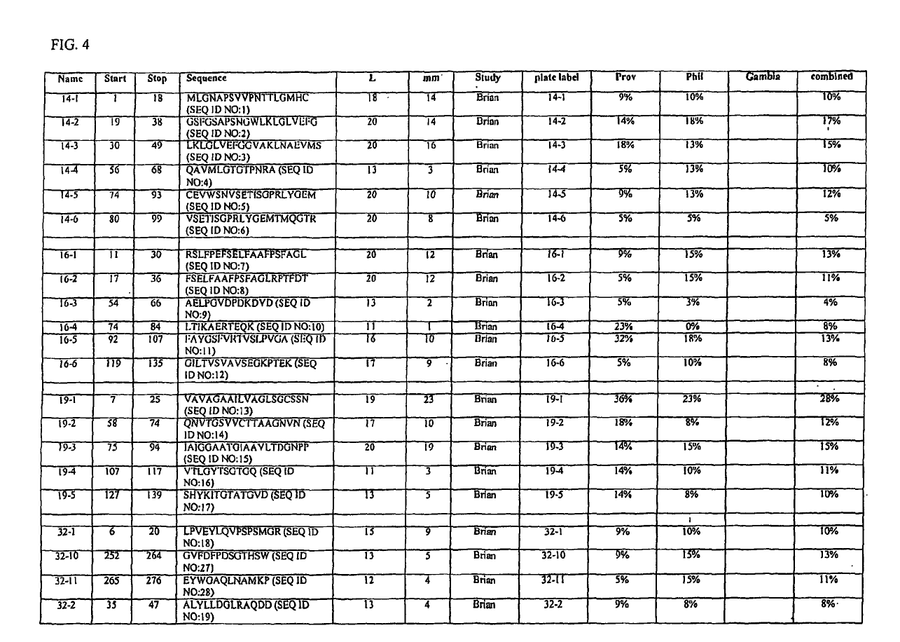

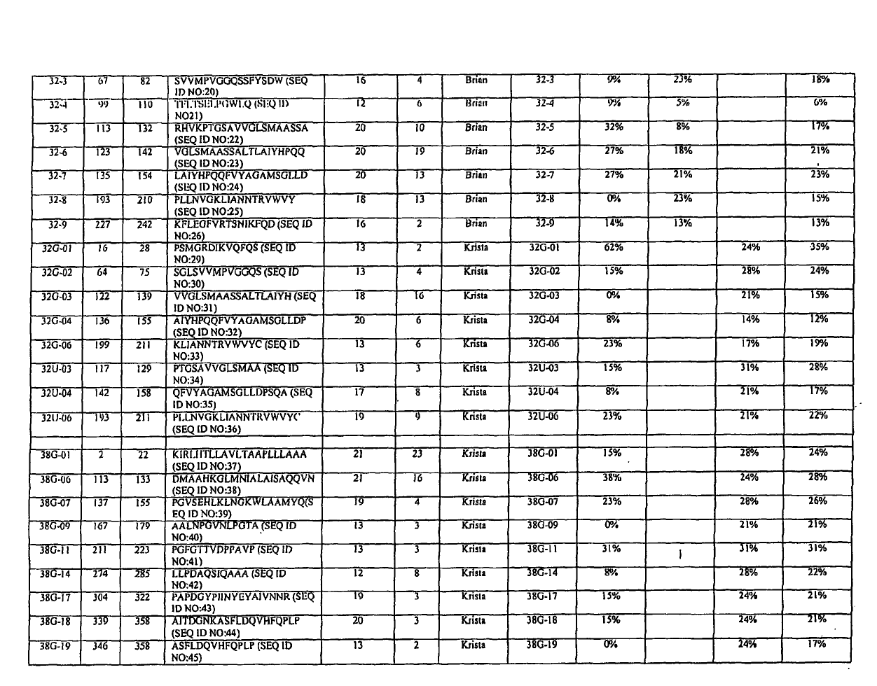

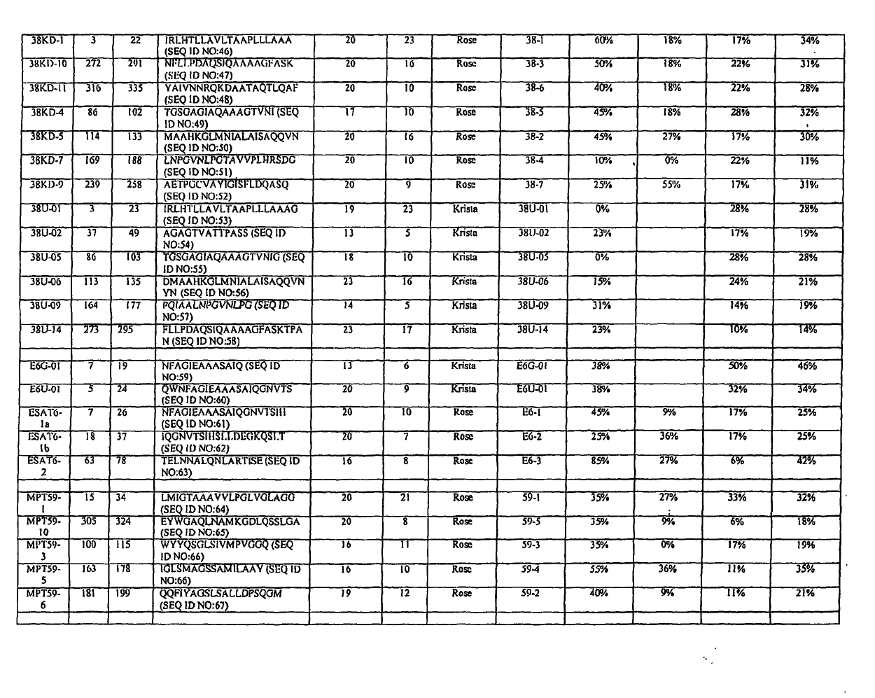

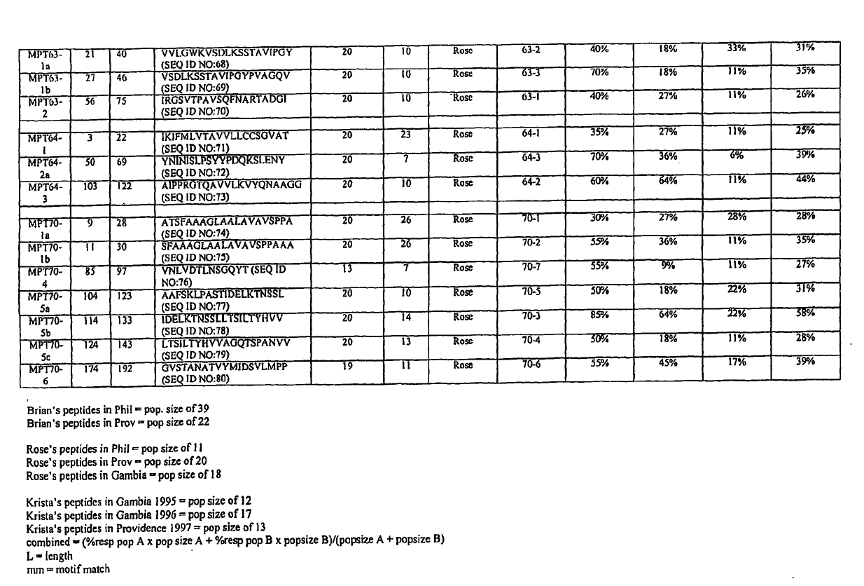

In order to determine whether peptides with multiple motif matches were more likely to induce a response in multiple Mtb-immune subjects, the peptides were dichotomized by number of motif matches (0 to 4 and ≧5) and by number of responders (0 to 5 and ≧6), using the final list of ML 0595 motifs (Table 3). Peptides with at least five motif matches were more likely to induce a response in 8 or more subjects (71%) than peptides with fewer than five motif matches (14%) (RR 5.0, 95% CI 1-7 to 14). A regression analysis also demonstrates this relationship (R²=42%) (FIG. 3).

### EXAMPLE 3

### Measuring Individual Responses to Other Peptides Containing Motifs Matched to Subjects' HLA-DR Allele

HLA Variation in Populations. The distribution of MHC alleles varies from population to population. In general, the MHC-peptide (epitope) interaction is governed by the sequence of the peptide each MHC has its own constraints, which can be described as a pattern, or motif, characterizing the set of peptides that can bind in the binding groove of the MHC. While the distribution of MHC in populations inhabiting different regions of the world may restrict, to some extent, the relevance of selected epitopes in different human populations, means to surmount this difficulty have been proposed. For example, identification of epitopes that may be recognized in the context of more than one MHC, such as “promiscuous” or “clustered” MHC binding regions, may permit the development of vaccines that effectively protect genetically diverse human populations.

HLA typing. At the time of PBMC isolation, a small sample of cells from some subjects was suspended in cell freezing medium (Sigma) and stored in liquid nitrogen. Using supernatant from the immortalized B95.8 cell line (ATCC, Rockville, Md.), EBV-transformed B cell lines were generated from thawed PBMG (on occasion, fresh PBMC were used in this step). Cell lines were sent to the Rhode Island Blood Center, Providence, R.I., where HLA-DR typing by the polymerase chain reaction (PCR) technique was performed for 18 of the 40 Mtb immunes, nine from the Providence cohort, and nine from the Philippine cohort. The IRA-DR type of each subject is listed in Table 4. Only DR typing was performed, as most published motifs included in EpiMer ML059S motif lists belonged to the DR subtypes. Twenty-two of the subjects were not HLA-DR typed owing to insufficient cells available after freezing.

Statistical analysis. All statistical analyses were performed in Microsoft Excel v4.0 or v5.0 Microsoft Corporation, Redmond, Wash.) using a Macintosh Quadra 650 or a PowerMacintosh 7100 (Apple Computer, Inc., Cupertino, Calif.). The relative risk (RR) of response and the 95% confidence intervals (CI) for this ratio were calculated using an Excel v4.0 spreadsheet.

Results—HLA typing. For the 18 HLA-DR typed subjects, there was an increased likelihood of observing a proliferative response to a peptide which contained a motif matched for the subject's allele, compared to cases where the peptide did not contain a motif matched for the subjects allele. When stratified by subject, and then pooled by the Mantel-Haenzel method, RR=1.5 (95% CI 0.9 to 2.5). Some of the discordant positive responses (46 cases) may have been due to presentation by a DP or DQ allele that was not represented in the EpiMer ML 0595 database. Some of the discordant negative responses (120 cases) may have been due to inaccurate motifs, inhibition of peptide binding by non-anchor residues, absence of T cells recognizing that particular peptide, insensitivity of the assay system, or the method of analysis.

### EXAMPLE 4

### EpiMer Algorithm

This study provides an in vitro assessment of EpiMer predictions for Mycobacterium tuberculosis (Mtb) vaccine candidate peptides. EpiMer, and other MHC-binding motif-based algorithms, may be useful methods for identifying “promiscuous” peptides, which can be recognized by a higher number of individuals in outbred human populations. The cost and time savings of this method over the traditional overlapping approach are substantial, and this method may eventually contribute to the development of a novel sub-unit vaccine against Mtb.

The EpiMer algorithm is designed to identify peptides that have the ability to bind to multiple MHC alleles. Peptides with this property have been described as ‘promiscuous’ or ‘universal’ epitopes. In this study, the number of study subjects who had a significant proliferative response to the peptides was associated with the number of MHC-binding motifs contained within the predicted peptide (RR=5.0, 95% CI 1.7 to 14). However, the association between number of MHC-binding motifs contained within a peptide and response to the peptide was not absolute, as demonstrated by several peptides which contained a large number of MHC-binding motifs but stimulated in vitro response in only a few of the study subjects, and vice versa (32-3 and 32-9 respectively).

One reason for this may be that peptides which contain multiple anchor based binding motifs may also contain amino acids that have other features (such as bulky or charged side chains, or cleavage sites) which inhibit the peptides from binding to certain MHC molecules (Boehncke, et al., J Immunol 150:331 (1993); Ruppert, et al., Cell 74:205 (1993)). In contrast, peptides that contained no motif matches according to our ML 0595 list may indeed contain MHC-binding motifs or ligands that have yet to be described or included.

At the time of epitope prediction for the studies described, the MHC-binding motif database consisted of a total of 15 distinct human motifs. Later, we found that 26 human class II MHC-binding motifs are utilized by EpiMer ML 0595 (Table 3). Some motifs that were used by EpiMer at the time of epitope prediction have since been shown to be inaccurate predictors of MHC-binding, and as such, are no longer included for use by the EpiMer algorithm. As more MHC-binding motifs are identified, and existing motifs are refined through further study, the algorithm's predictive capacity is expected to improve.

For many of the subjects in the Mtb immune group, the date of PPD skin-test conversion was not known. This leaves open the possibility that some of these subjects might have been exposed to Mtb many years prior to the collection of their PBMC, while others may have been exposed more recently, resulting in a range of immune responses in our subject cohort as measured in the T cell proliferation assay. Within the Mtb näive control group, four of the Mtb näive subjects showed proliferative responses to five or more peptides as well as an in vitro response to PPD. These responses could be due to subclinical (and PPD skin-test negative) infection with Mtb, or to exposure to environmental mycobacteria, leading to cross-reactive proliferative responses to shared antigens (in the case of PPD) or to shared regions of protein sequences (in the case of responses to specific peptides) (Stanford, et al., J Hyg Lond 76:205 (1976)). Furthermore, the 14 kDa protein has been shown to be unique to Mtb; therefore proliferative responses seen in five Mtb naive individuals to peptides derived from this antigen are difficult to explain, unless subclinical exposure had occurred, or the particular peptide used in this assay is similar to T cell epitopes derived from other antigenic proteins. Until better tests can be developed to confirm latent Mtb infection, it is difficult to determine how to classify PPD skin test positive individuals who have no known date of exposure to Mtb infection and few in vitro responses to Mtb antigens.

Similarly, non-responsiveness to the tetanus toxoid antigen can probably be explained by the fact that immunity derived by immunization with TT is not life-long, and that such acquired immunity wanes without frequent boosting (Gergen, et al., New Eng J Med 332:761 (1995)).

### OTHER EMBODIMENTS

The details of one or more embodiments of the invention are set forth in the accompanying description above. Although any methods and materials similar or equivalent to those described herein can be used in the practice or testing of the present invention, the preferred methods and materials have been described. Other features, objects, and advantages of the invention will be apparent from the description and from the claims. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but only to the claims appended hereto.

We claim:

1. A vaccine comprising:
a Mycobacterium tuberculosis (Mtb) vaccine candidate peptide containing an amino acid sequence selected from the group consisting of SEQ ID NOs:1-28, in an immunologically acceptable excipient.

2. The vaccine of claim 1, wherein the peptide is between 8 amino acids and 50 amino acids in length.

3. The vaccine of claim 1, wherein the Mtb vaccine candidate peptide has an amino acid sequence selected from the group consisting of SEQ ID NOs:1-28.

4. The vaccine of claim 1, wherein the peptide is complexed to a carrier protein.

5. The vaccine of claim 1, wherein the peptide is a recombinant fusion protein.

6. The vaccine of claim 1, wherein the excipient is an adjuvant.

7. A recombinant Mycobacterium tuberculosis (Mtb) vaccine candidate peptide, comprising:
a peptide containing an amino acid sequence selected from the group consisting of SEQ ID NOs:1-28, wherein the peptide is expressed from a recombinant polynucleotide.

8. The recombinant peptide of claim 7, wherein the recombinant polynucleotide is a naked DNA vaccine.

9. A method for inducing an anti-Mycobacterium tuberculosis (anti-Mtb) immune response, comprising:
administering to a mammalian subject a Mtb vaccine candidate peptide containing an amino acid sequence selected from the consisting of SEQ ID NOs:1-28.

10. The method of claim 9, wherein the induction of an anti-Mtb immune response is the raising of an anti-Mtb antibody.

11. The method of claim 9, wherein the mammalian subject is a human.

12. The method of claim 9, wherein the administration is selected from the group consisting of orally, topically, parenterally, by viral infection, and intravascularly.


SEQUENCE LISTING (81 sequences)

SEQ ID NO: 1 - protein, 18 aa, Mycobacterium tuberculosis
MLGNAPSVVPNTTLGMHC

SEQ ID NO: 2 - protein, 20 aa, Mycobacterium tuberculosis
GSFGSAPSNGWLKLGLVEFG

SEQ ID NO: 3 - protein, 20 aa, Mycobacterium tuberculosis
LKLGLVEFGGVAKLNAEVMS

SEQ ID NO: 4 - protein, 13 aa, Mycobacterium tuberculosis
QAVMLGTGTPNRA

SEQ ID NO: 5 - protein, 20 aa, Mycobacterium tuberculosis
CEVWSNVSETISGPRLYGEM

SEQ ID NO: 6 - protein, 20 aa, Mycobacterium tuberculosis
VSETISGPRLYGEMTMQGTR

SEQ ID NO: 7 - protein, 20 aa, Mycobacterium tuberculosis
RSLFPEFSELFAAFPSFAGL

SEQ ID NO: 8 - protein, 20 aa, Mycobacterium tuberculosis
FSELFAAFPSFAGLRPTFDT

SEQ ID NO: 9 - protein, 13 aa, Mycobacterium tuberculosis
AELPGVDPDKDVD

SEQ ID NO: 10 - protein, 11 aa, Mycobacterium tuberculosis
LTIKAERTEQK

SEQ ID NO: 11 - protein, 16 aa, Mycobacterium tuberculosis
FAYGSFVRTVSLPVGA

SEQ ID NO: 12 - protein, 17 aa, Mycobacterium tuberculosis
GILTVSVAVSEGKPTEK

SEQ ID NO: 13 - protein, 19 aa, Mycobacterium tuberculosis
VAVAGAAILVAGLSGCSSN

SEQ ID NO: 14 - protein, 17 aa, Mycobacterium tuberculosis
QNVTGSVVCTTAAGNVN

SEQ ID NO: 15 - protein, 20 aa, Mycobacterium tuberculosis
IAIGGAATGIAAVLTDGNPP

SEQ ID NO: 16 - protein, 11 aa, Mycobacterium tuberculosis
VTLGYTSGTGQ

SEQ ID NO: 17 - protein, 13 aa, Mycobacterium tuberculosis
SHYKITGTATGVD

SEQ ID NO: 18 - protein, 15 aa, Mycobacterium tuberculosis
LPVEYLQVPSPSMGR

SEQ ID NO: 19 - protein, 13 aa, Mycobacterium tuberculosis
ALYLLDGLRAQDD

SEQ ID NO: 20 - protein, 16 aa, Mycobacterium tuberculosis
SVVMPVGGQSSFYSDW

SEQ ID NO: 21 - protein, 12 aa, Mycobacterium tuberculosis
TFLTSELPGWLQ

SEQ ID NO: 22 - protein, 20 aa, Mycobacterium tuberculosis
RHVKPTGSAVVGLSMAASSA

SEQ ID NO: 23 - protein, 20 aa, Mycobacterium tuberculosis
VGLSMAASSALTLAIYHPQQ

SEQ ID NO: 24 - protein, 20 aa, Mycobacterium tuberculosis
LAIYHPQQFVYAGAMSGLLD

SEQ ID NO: 25 - protein, 18 aa, Mycobacterium tuberculosis
PLLNVGKLIANNTRVWVY

SEQ ID NO: 26 - protein, 16 aa, Mycobacterium tuberculosis
KFLEGFVRTSNIKFQD

SEQ ID NO: 27 - protein, 13 aa, Mycobacterium tuberculosis
GVFDFPDSGTHSW

SEQ ID NO: 28 - protein, 12 aa, Mycobacterium tuberculosis
EYWGAQLNAMKP

SEQ ID NO: 29 - protein, 13 aa, Mycobacterium tuberculosis
PSMGRDIKVQFQS

SEQ ID NO: 30 - protein, 13 aa, Mycobacterium tuberculosis
SGLSVVMPVGGQS

SEQ ID NO: 31 - protein, 18 aa, Mycobacterium tuberculosis
VVGLSMAASSALTLAIYH

SEQ ID NO: 32 - protein, 20 aa, Mycobacterium tuberculosis
AIYHPQQFVYAGAMSGLLDP

SEQ ID NO: 33 - protein, 13 aa, Mycobacterium tuberculosis
KLIANNTRVWVYC

SEQ ID NO: 34 - protein, 13 aa, Mycobacterium tuberculosis
PTGSAVVGLSMAA

SEQ ID NO: 35 - protein, 17 aa, Mycobacterium tuberculosis
QFVYAGAMSGLLDPSQA

SEQ ID NO: 36 - protein, 19 aa, Mycobacterium tuberculosis
PLLNVGKLIANNTRVWVYC

SEQ ID NO: 37 - protein, 21 aa, Mycobacterium tuberculosis
KIRLHTLLAVLTAAPLLLAAA

SEQ ID NO: 38 - protein, 21 aa, Mycobacterium tuberculosis
DMAAHKGLMNIALAISAQQVN

SEQ ID NO: 39 - protein, 19 aa, Mycobacterium tuberculosis
PGVSEHLKLNGKWLAAMYQ

SEQ ID NO: 40 - protein, 13 aa, Mycobacterium tuberculosis
AALNPGVNLPGTA

SEQ ID NO: 41 - protein, 13 aa, Mycobacterium tuberculosis
PGFGTTVDPPAVP

SEQ ID NO: 42 - protein, 12 aa, Mycobacterium tuberculosis
LLPDAQSIQAAA

SEQ ID NO: 43 - protein, 19 aa, Mycobacterium tuberculosis
PAPDGYPIINYEYAIVNNR

SEQ ID NO: 44 - protein, 20 aa, Mycobacterium tuberculosis
AITDGNKASFLDQVHFQPLP

SEQ ID NO: 45 - protein, 13 aa, Mycobacterium tuberculosis
ASFLDQVHFQPLP

SEQ ID NO: 46 - protein, 20 aa, Mycobacterium tuberculosis
IRLHTLLAVLTAAPLLLAAA

SEQ ID NO: 47 - protein, 20 aa, Mycobacterium tuberculosis
NFLLPDAQSIQAAAAGFASK

SEQ ID NO: 48 - protein, 20 aa, Mycobacterium tuberculosis
YAIVNNRQKDAATAQTLQAF

SEQ ID NO: 49 - protein, 17 aa, Mycobacterium tuberculosis
TGSGAGIAQAAAGTVNI

SEQ ID NO: 50 - protein, 20 aa, Mycobacterium tuberculosis
MAAHKGLMNIALAISAQQVN

SEQ ID NO: 51 - protein, 20 aa, Mycobacterium tuberculosis
LNPGVNLPGTAVVPLHRSDG

SEQ ID NO: 52 - protein, 20 aa, Mycobacterium tuberculosis
AETPGCVAYIGISFLDQASQ

SEQ ID NO: 53 - protein, 21 aa, Mycobacterium tuberculosis
IRLHTLLAVLTAAPLLLAAAG

SEQ ID NO: 54 - protein, 13 aa, Mycobacterium tuberculosis
AGAGTVATTPASS

SEQ ID NO: 55 - protein, 18 aa, Mycobacterium tuberculosis
TGSGAGIAQAAAGTVNIG

SEQ ID NO: 56 - protein, 23 aa, Mycobacterium tuberculosis
DMAAHKGLMNIALAISAQQVNYN

SEQ ID NO: 57 - protein, 14 aa, Mycobacterium tuberculosis
PQIAALNPGVNLPG

SEQ ID NO: 58 - protein, 23 aa, Mycobacterium tuberculosis
FLLPDAQSIQAAAAGFASKTPAN

SEQ ID NO: 59 - protein, 13 aa, Mycobacterium tuberculosis
NFAGIEAAASAIQ

SEQ ID NO: 60 - protein, 20 aa, Mycobacterium tuberculosis
QWNFAGIEAAASAIQGNVTS

SEQ ID NO: 61 - protein, 20 aa, Mycobacterium tuberculosis
NFAGIEAAASAIQGNVTSIH

SEQ ID NO: 62 - protein, 20 aa, Mycobacterium tuberculosis
IQGNVTSIHSLLDEGKQSLT

SEQ ID NO: 63 - protein, 16 aa, Mycobacterium tuberculosis
TELNNALQNLARTISE

SEQ ID NO: 64 - protein, 20 aa, Mycobacterium tuberculosis
LMIGTAAAVVLPGLVGLAGG

SEQ ID NO: 65 - protein, 20 aa, Mycobacterium tuberculosis
EYWGAQLNAMKGDLQSSLGA

SEQ ID NO: 66 - protein, 16 aa, Mycobacterium tuberculosis
WYYQSGLSIVMPVGGQ

SEQ ID NO: 67 - protein, 19 aa, Mycobacterium tuberculosis
QQFIYAGSLSALLDPSQGM

SEQ ID NO: 68 - protein, 20 aa, Mycobacterium tuberculosis
VVLGWKVSDLKSSTAVIPGY

SEQ ID NO: 69 - protein, 20 aa, Mycobacterium tuberculosis
VSDLKSSTAVIPGYPVAGQV

SEQ ID NO: 70 - protein, 20 aa, Mycobacterium tuberculosis
IRGSVTPAVSQFNARTADGI

SEQ ID NO: 71 - protein, 20 aa, Mycobacterium tuberculosis
IKIFMLVTAVVLLCCSGVAT

SEQ ID NO: 72 - protein, 20 aa, Mycobacterium tuberculosis
YNINISLPSYYPDQKSLENY

SEQ ID NO: 73 - protein, 21 aa, Mycobacterium tuberculosis
AIPPRGTQAVVLKVYQNAAGG

SEQ ID NO: 74 - protein, 20 aa, Mycobacterium tuberculosis
ATSFAAAGLAALAVAVSPPA

SEQ ID NO: 75 - protein, 20 aa, Mycobacterium tuberculosis
SFAAAGLAALAVAVSPPAAA

SEQ ID NO: 76 - protein, 13 aa, Mycobacterium tuberculosis
VNLVDTLNSGQYT

SEQ ID NO: 77 - protein, 20 aa, Mycobacterium tuberculosis
AAFSKLPASTIDELKTNSSL

SEQ ID NO: 78 - protein, 20 aa, Mycobacterium tuberculosis
IDELKTNSSLLTSILTYHVV

SEQ ID NO: 79 - protein, 20 aa, Mycobacterium tuberculosis
LTSILTYHVVAGQTSPANVV

SEQ ID NO: 80 - protein, 19 aa, Mycobacterium tuberculosis
GVSTANATVYMIDSVLMPP

SEQ ID NO: 81 - protein, 16 aa, Mycobacterium tuberculosis
IGLSMAGSSAMILAAY